(19) United States
(12) Patent Application Publication
(10) Pub. No.: US 2026/0198768 A1
(43) Pub. Date: Jul. 16, 2026

(54) ORAL ENDOSCOPIC SCANNING DEVICE

(71) Applicant: Shanghai Dian-Tai Medical Technology Co., Ltd., Shanghai (CN)

(72) Inventors: YE TIAN, Shanghai (CN); XIONGJIE YANG, Shanghai (CN)

(21) Appl. No.: 19/539,182

(22) Filed: Feb. 13, 2026

(30) Foreign Application Priority Data
Sep. 4, 2025 (CN) 202511264077.4

(51) Int. Cl. A61B 1/247; A61B 1/00; A61B 1/05; A61B 1/06; A61B 90/00

(52) U.S. Cl. CPC A61B 1/247; A61B 1/00016; A61B 1/00034; A61B 1/00066; A61B 1/0008; A61B 1/00128; A61B 1/05; A61B 1/0607; A61B 1/0684; A61B 90/03

(57) ABSTRACT

An oral endoscopic scanning device is provided, including a handheld assembly, an imaging head, and a turning structure. The imaging head includes a main imaging base, at least two groups of cameras, and two elastic clamping portions respectively arranged on two sides of the main imaging base. The cameras surround an imaging region and are disposed on the main imaging base and/or a connection arm. On the same horizontal plane projection, two adjacent cameras are disposed at an angle, so that capturing ranges of the two adjacent cameras partially overlap. The turning structure achieves multi-angle rotation of the imaging head. A plurality of cameras work cooperatively, so that an image acquisition range is significantly enlarged, and a user feels more flexible and more comfortable during operation.

### CROSS-REFERENCE TO RELATED APPLICATIONS

This application claims priority to Chinese Patent Application No. 202511264077.4, filed on 09/04/2025, which is hereby incorporated by reference in its entirety.

### TECHNICAL FIELD

The present disclosure relates to the field of oral medical instruments, and in particular, to an oral endoscopic scanning device.

### BACKGROUND

Consumer-grade oral endoscope systems have gained increasing importance with the growing popularity of household health products. Consumers have a significantly increasing awareness of the oral health and expect to conveniently examine and record the oral health by themselves in daily life. Compared with a traditional manual operation, portable intelligent oral imaging devices can provide clear real-time images and have a basic data analysis function. It helps a user to intuitively know dental conditions (such as dental calculus and dental caries) and gum health states, to promptly find out issues and take preventive measures.

CN113349721B is taken as an example. This design integrates a lens frame, a rotating wind wheel, and a mirror body structure. Saliva ejection suction power is used to drive the rotating wind wheel, which drives a mirror body to rotate, thus achieving defogging and self-cleaning functions. Although this structure solves the difficulty in mirror cleaning, it is only provided with a single oral endoscopic scanning device, which lacks a multi-angle synchronous imaging capability. Consequently, in clinical applications, this structure is still limited by the field of view, making it difficult to meet a requirement of a doctor for multi-directional synchronous observation of an oral cavity.

CN217310246U is taken as an example. A patient needs to grasp the oral endoscopic scanning device and put it into the oral cavity. A user has to repeatedly rotate a handle to adjust a photographing position. This device has a limited imaging angle, leading to insufficient photographing stability, and it is necessary to frequently move a probe to observe a target region. This not only prolongs the operation time, but also exacerbates the discomfort of the patient.

In summary, oral endoscopes on the current market predominantly employ the single oral endoscopic scanning device structure. There are the following notable shortcomings: 1. Limited imaging angle, namely, it is difficult to achieve comprehensive observation in a complex oral environment, and the position of the probe needs to be often repeatedly adjusted, thus increasing the discomfort of the patient and prolonging the operation time. 2. Pronounced functional limitation, namely most devices only support real-time image viewing and cannot meet advanced functions such as three-dimensional reconstruction, thereby constraining the fulfillment of complex diagnostic requirements.

### SUMMARY

To overcome the above shortcomings in the existing technology, the present disclosure provides an oral endoscopic scanning device. In the present disclosure, a plurality of cameras work cooperatively, so that an image acquisition range is significantly enlarged, the repeated operation time is shortened, and the time required by scanning is greatly shortened. By a rotatable head portion and an elastic clamping structure, a user feels more flexible and more comfortable during operation. The oral endoscopic scanning device is particularly suitable for a scenario for long-time use.

To achieve the above objectives, the present disclosure provides an oral endoscopic scanning device, including:

a handheld assembly, where a control circuit board is disposed within the handheld assembly;

an imaging head, where an imaging head includes a main imaging base, at least two groups of cameras, and two elastic clamping portions respectively arranged on two sides of the main imaging base; connection arms are disposed between the main imaging base and the elastic clamping portions; the cameras surround an imaging region and are disposed on the main imaging base and/or the connection arms; on the same horizontal plane projection, two adjacent cameras are disposed at an angle, so that capturing ranges of the two adjacent cameras partially overlap, and after overlapping, the capturing ranges of all the cameras cover the imaging region; the cameras are connected to the control circuit board through data wires provided, to implement image transmission; and

a turning structure, where the turning structure further includes a limiting structure; the limiting structure includes a first limiting convex ring disposed at a periphery of the connection through hole; a second limiting convex ring fitting with the first limiting convex ring is also arranged on one side of the connection ring body; and the main imaging base is connected to the handheld assembly through the turning structure to implement multi-angle rotation of the imaging head.

Further, an angle θ between the two adjacent cameras on the same horizontal plane projection is less than or equal to 90°. By forming an angle between two adjacent cameras, arrangement of the cameras at different angles is achieved to ensure that the cameras capture images from various perspectives. Since the capturing ranges of the cameras overlap, the cameras can completely cover the imaging region and can cover teeth placed in the imaging region in a plurality of directions for imaging.

Further, the connection arms are arranged on two sides of the main imaging base in parallel, or the connection arms downwards slantways extend respectively from the two sides of the main imaging base. The tilting of the connection arms can adapt to the mounting of the cameras, which facilitates the setting of the angles of the cameras. Certainly, tilting angles of the connection arms can be adjusted based on an actual need to cooperate with the cameras to implement optimal viewing angle coverage in different oral structures. This further optimizes the imaging quality. In addition, the connection arms are made of a high-strength material. This ensures the structural stability, enhances the durability of the device, and ensures that accurate imaging performance can still be maintained during long-time use.

Further, first bent portions are arranged between the elastic clamping portions and the connection arms, to cause the two elastic clamping portions to be horizontally opposite to each other and form, between the two elastic clamping portions, a clamping opening for passing through an alveolar cavity. The elastic clamping portions are components made of silicone or other elastic materials with the same performance. This greatly enhances the comprehensiveness and accuracy of diagnosis. Meanwhile, the adaptive fitting characteristic of the elastic clamping portions further enhances the applicability and stability of the device in different oral shapes, so that the optimal imaging quality can be achieved in each examination.

Further, light-emitting diode (LED) light groups are arranged at a periphery of the main imaging base and/or peripheries of the cameras. By the arrangement of the LED light groups, it ensures a lighting environment inside an oral cavity. The brightness of LED lights can be adjusted to meet different oral environments and an observation need.

Further, the imaging head further includes a connector. A connection slot with an opening in one side is formed in the main imaging base. The connector is plugged into the connection slot for locking. The turning structure is arranged on the connector. By the arrangement of the connector and the connection slot, the imaging head can be quickly replaced, and the maintenance efficiency and usage convenience of the device are enhanced.

Further, the turning structure includes a connection ring body protruding from the connector. A connection through hole is formed in one end of the handheld assembly away from the control circuit board. The connection ring body passes through and is connected into the connection through hole in a threaded manner, to implement rotation of the imaging head. The turning structure can implement the rotation of the imaging head, so as to satisfy that the imaging head moves along a dental arch curve from a posterior tooth on one side to a posterior tooth on the other side to ensure observation without a dead angle. This design enhances the diagnostic accuracy and greatly lowers the operation difficulty, so that an oral examination process is more efficient and comfortable, and the oral endoscopic scanning device is applicable to precision diagnosis and treatment in various types of complex oral environments.

Further, the turning structure further includes a limiting structure. The limiting structure includes a first limiting convex ring disposed at a periphery of the connection through hole. A second limiting convex ring fitting with the first limiting convex ring is also arranged on one side of the connection ring body. An angle limiting function is integrated to the turning structure to limit a rotation angle within a specific range to prevent unconstrained 360° free rotation, thus avoiding damage to or mis-operation on the imaging head caused by excessive rotation in an operation process, ensuring that each rotation is within a safe and controllable range, and further enhancing the stability and service life of the device.

Further, the oral endoscopic scanning device further includes a support. The support is perpendicularly arranged on the main imaging base. Two supports may be provided. During use, the supports can resist against top surfaces of teeth, as a height limitation, to ensure the stability of a relative position.

Further, an elastic abutment portion is arranged at a bottom of the support. The elastic abutment portion can be made of a silicone material or other elastic materials with the same effect, so that the support can slide on tooth surfaces more smoothly.

Further, a physical mark and a standard reference substance are arranged on the support and/or the elastic clamping portion. A physical mark with a known length (such as a portion of a standard scale) is integrated onto the support and/or the elastic clamping portions, and is ensured to be clearly visible, so that precise size and color calibration can be provided for model reconstruction, thus enhancing the diagnostic reliability. It is convenient for quick positioning by a doctor using the physical mark, and consistent reference can also be provided during multi-angle imaging to ensure the accuracy of data acquisition, thus enhancing the accuracy of three-dimensional model reconstruction and providing a solid basis for formulation of subsequent treatment schemes.

Compared with the existing technology, the present disclosure has the following advantages:

By the arrangement of the at least two cameras on the main imaging base, the present disclosure can flexibly increase or decrease the number of cameras, as long as at least two cameras are provided. The cameras are mounted on the main imaging base at different angles and are spaced apart to ensure that the angle θ is formed between two adjacent cameras, and a photographing region of adjacent cameras overlap with each other. Therefore, the photographing region formed by the plurality of cameras can completely cover a specific range of the teeth. This multi-viewing-angle cooperation mechanism can capture oral details in all angles, enhance the image stitching precision, and eliminate photographing blind spots.

In addition, the present disclosure is provided with the support. During use, the supports can abut against the top surfaces of the teeth to limit a height. The physical mark and the reference substance that are arranged on the support and/or the elastic clamping portions can provide a precise size basis and color calibration benchmark for the model reconstruction, thus significantly enhancing the diagnostic reliability. These physical marks provide direct and reliable depth information and calibration benchmark, which can effectively correct a proportional scaling relationship of image data and ensure that true size displaying of oral model reconstruction is implemented in subsequent processing (achieving 1:1 modeling accuracy). Meanwhile, a standard reference substance with a specific color can assist in calibration and correction of color deviations. This physical reference system integrated under an intraoral operation condition significantly enhances the accuracy of size quantification and visual presentation of an oral model constructed based on image information.

The elastic clamping portions are clamped on the alveolar bone during use, and a movement manner for the elastic clamping portions is as follows: a user applies a force to the device from the posterior teeth to cause the device to move along the dental arch curve from the posterior tooth on one side to a front tooth and then move from the front tooth to the posterior tooth on the other side. In this process, the plurality of cameras continuously take videos to obtain multi-angle images around the teeth by one circle. This design achieves all-round oral observation, and provides a doctor with dynamic and three-dimensional oral images through real-time integration of video data, thus significantly enhancing the comprehensiveness and accuracy of diagnosis. Meanwhile, the adaptive fitting characteristic of the elastic clamping portions further enhances the applicability and stability of the device in different oral shapes, so that the optimal imaging quality can be achieved in each examination.

### BRIEF DESCRIPTION OF THE DRAWINGS

To describe the technology in the embodiments of the present disclosure or in the related art more clearly, the following briefly introduces the accompanying drawings for describing the embodiments or the related art. Apparently, the accompanying drawings in the following description show some embodiments of the present disclosure, and a person of ordinary skill in the art may still derive other drawings from the accompanying drawings without creative efforts.

FIG. 1 is a schematic diagram of a structure of an oral endoscopic scanning device according to the present disclosure;

FIG. 2 is an exploded view of FIG. 1;

FIG. 3 is a schematic diagram of a structure of an imaging head according to the present disclosure;

FIG. 4 is a schematic diagram of structures of a main imaging base and a connector according to the present disclosure;

FIG. 5 is a schematic diagram of a structure of a connection through hole of a handheld assembly according to the present disclosure;

FIG. 6 is a schematic diagram of structures of a camera and LED light groups according to the present disclosure;

FIG. 7 is a schematic diagram of assembling of a main imaging base, connection arms, and elastic clamping portions according to the present disclosure;

FIG. 8 is a schematic diagram of a main imaging base, connection arms, and elastic clamping portions on the same horizontal plane projection according to another implementation of the present disclosure; and

FIG. 9 is a schematic diagram of a main imaging base, connection arms, and elastic clamping portions on the same horizontal plane projection according to still another implementation of the present disclosure.

In the drawings:

1: handheld assembly; 11: accommodating space; 12: control circuit board; 121: control button; 122: lithium battery; 123: Type-C interface; 124: wireless module; 125: LED indicator light; 13: connection through hole; 131: bearing; 14: second bent portion; 15: recess structure; 2: imaging head; 21: main imaging base; 211: connection slot; 212: connection arm; 213: imaging region; 214: mounting groove body; 215: first bent portion; 22: connector; 23: camera; 231: camera housing; 232: flexible printed circuit (FPC) flexible flat cable; 24: elastic clamping portion; 241: clamping opening; 3: turning structure; 31: connection ring body; 32: first limiting convex ring; 33: second limiting convex ring; 4: LED light group; and 5: support.

### DETAILED DESCRIPTION OF THE EMBODIMENTS

The technology in this implementation of the present disclosure is clearly and completely described below with reference to the accompanying drawings in this implementation of the present disclosure. Apparently, the described implementation is one implementation, rather than all implementations of the present disclosure. All other implementations obtained by a person of ordinary skill in the art based on this implementation of present disclosure without making creative efforts shall fall within the protection scope of present disclosure.

It should be noted that all directional indications (such as up, down, left, right, front, back, ...) involved in the embodiments of the present disclosure are only used to explain the relative positional relationship, motion states, and the like between various components in specific postures (as shown in the figures). If the specific postures change, the directional indications also change correspondingly.

In addition, if there are descriptions related to “first”, “second”, and the like in the embodiments of the present disclosure, the descriptions of “first”, “second”, and the like are only used for descriptive purposes and cannot be understood as indicating or implying their relative importance or implying the quantities of indicated technical features.

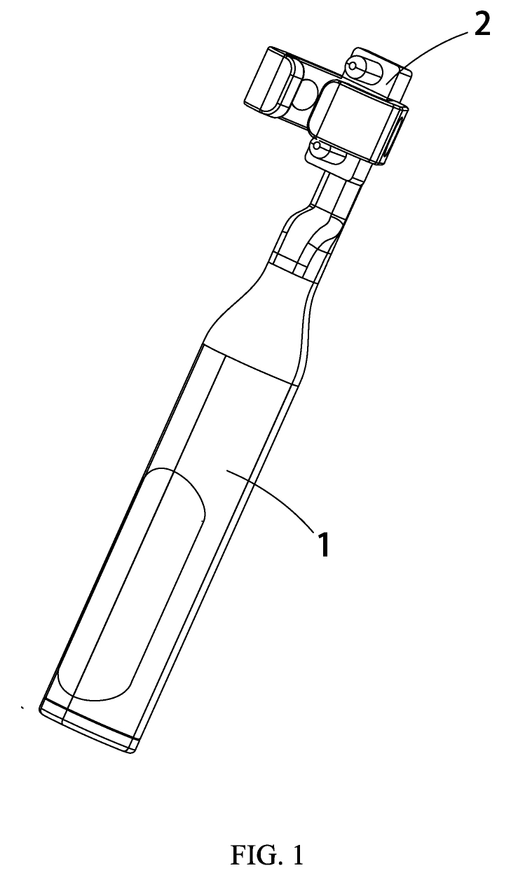

As shown in FIG. 1 to FIG. 9, the present disclosure provides an oral endoscopic scanning device, including a handheld assembly 1, an imaging head 2, and a turning structure 3.

As shown in FIG. 1, the handheld assembly 1 is of a long-strip-shaped handle structure. An accommodating space 11 is formed inside a rear end of the handheld assembly 1. A control circuit board 12 is fixed inside the accommodating space 11 through a screw. The control circuit board 12 is integrated with a control button 121, a lithium battery 122, a Type-C interface 123, a wireless module 124, and an LED indicator light 125. A plurality of keycaps are arranged on a surface of the handheld assembly 1. A keycap position corresponds to the control button 121 to achieve activation and deactivation of functions. The lithium battery 122 is configured to supply power.

In this embodiment, the control circuit board 12 is integrated with the Type-C interface 123 and the wireless module 124, thereby achieving wired and wireless dual-mode communications. The control circuit board 12 can be connected to a cable through the Type-C interface 123 for data transmission and to charge the lithium battery 122, and can also establish remote connection to a mobile device by using the wireless module 124. The wireless module 124 supports Bluetooth and Wi-Fi connection, and can be connected into a router network through Wi-Fi, thus facilitating real-time image viewing by other communication devices. It is preferred to employ a manner for establishing wireless connection to a router, and a user experience can be significantly enhanced.

Preferably, a dedicated application on a smartphone (such as an Android system, an iOS system, and a HarmonyOS system) can be used as a host computer, making it convenient for operation and use by a user in a home environment.

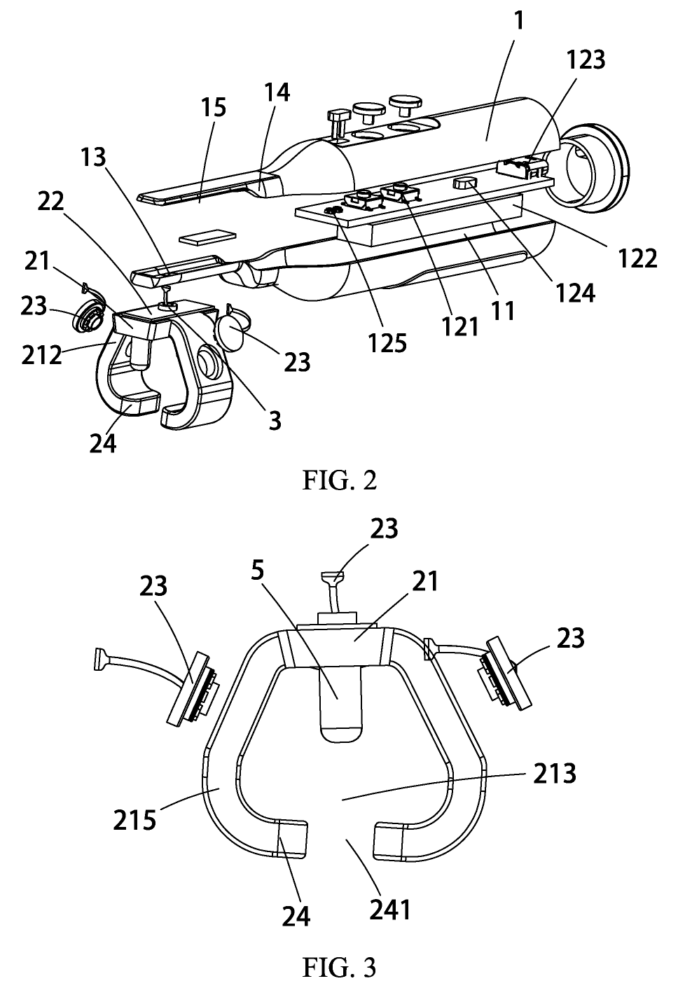

As shown in FIG. 2 to FIG. 4, the imaging head 2 of this embodiment includes a main imaging base 21, a connector 22, a camera 23, and two elastic clamping portions 24 respectively arranged on two sides of the main imaging base 21. Connection arms 212 are arranged between the main imaging base 21 and the elastic clamping portions 24. A connection slot 211 with an opening in one side is formed in an upper end of the main imaging base 21. The connection slot 211 is of a dovetail joint structure, and the connector 22 is also configured as a dovetail joint structure. During assembling, the connector 22 can be directly plugged into the connection slot 211 to achieve stable connection. Certainly, it is preferred to additionally provide a locking block to close the opening of the connection slot 211, so as to fix the connector 22 inside the connection slot 211 and prevent the connector 22 from falling off. For example, clamping holes can be formed in two sides of the opening. Clamping columns matching the clamping holes are disposed on the locking block. When locking is required, the clamping columns are directly plugged into the clamping holes. For another example, one side of the locking block is rotatably connected to the main imaging base 21, and another side of the locking block is fastened to the main imaging base 21. This can be set as needed as long as the connector 22 and the main imaging base 21 are locked.

The turning structure 3 includes a protruding connection ring body 31 arranged on the connector 22, and a connection through hole 13 located at one end (i.e. a front end) of the handheld assembly 1 away from the control circuit board 12. A bearing 131 is disposed within the connection through hole 13. The connection ring body 31 passes through and is connected into the bearing 131 of the connection through hole 13 to implement rotation of the imaging head 2. To satisfy the mounting of the main imaging base 21, a second bent portion 14 is arranged between a front end and a rear end of the handheld assembly 1, to form a recessed structure 15. After the connection ring body 31 is plugged into the connection through hole 13, the main imaging base 21 is exactly located within this recessed structure 15, making the overall structure more compact. The turning structure 3 supports the rotation of the imaging head 2, to cause the imaging head move along a dental arch curve from a posterior tooth on one side to a posterior tooth on the other side to ensure observation without a dead angle. This design enhances the diagnostic accuracy and significantly lowers the operation difficulty, so that an oral examination process is more efficient and comfortable, and the oral endoscopic scanning device is applicable to precision diagnosis and treatment in various types of complex oral environments.

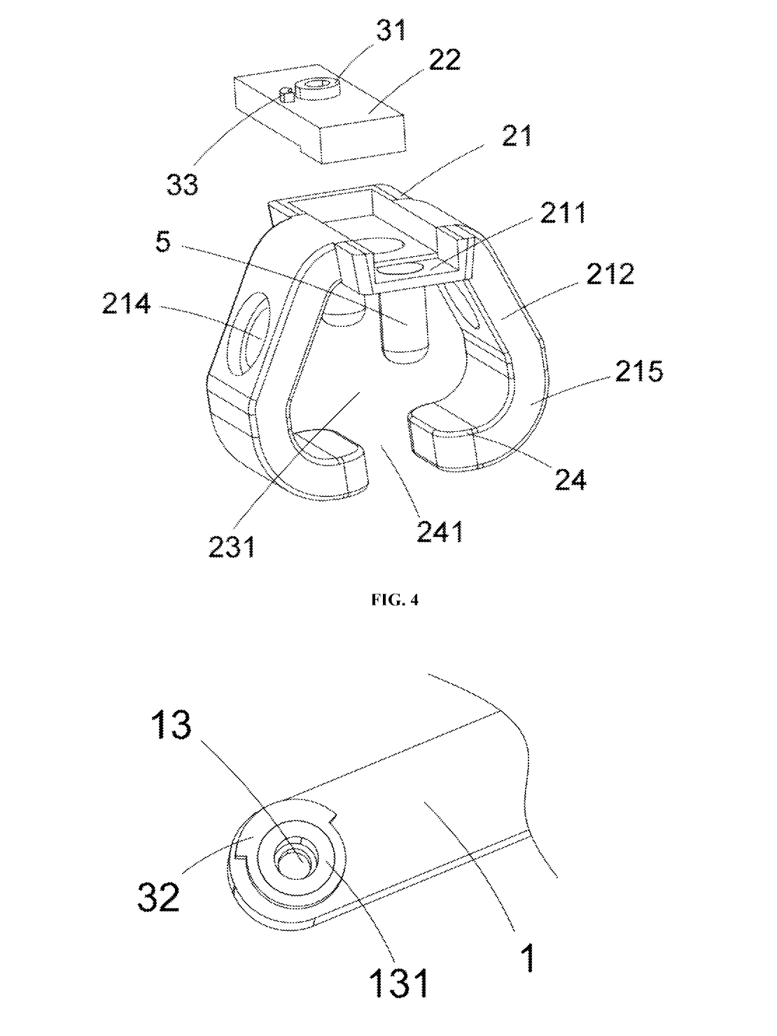

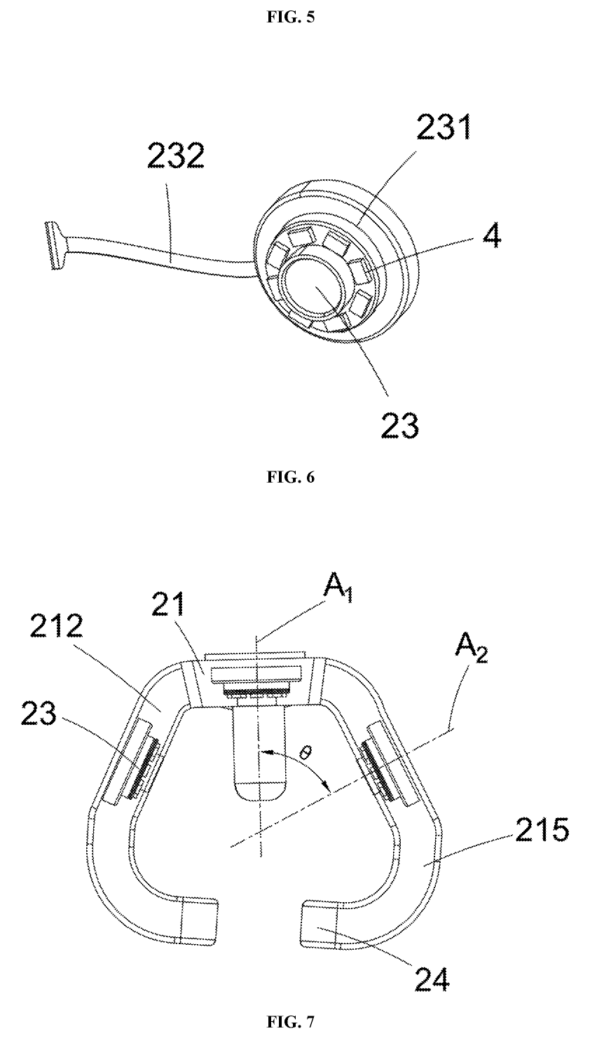

As shown in FIG. 4 and FIG. 5, the turning structure 3 further includes a limiting structure. The limiting structure includes a first limiting convex ring 32 disposed around the connection through hole 13. A second limiting convex ring 33 fitting with the first limiting convex ring 32 is also arranged on one side of the connection ring body 31. When the connection ring body 31 is plugged into the connection through hole 13, the first limiting convex ring 32 and the second limiting convex ring 33 are located on a coaxial arc, and the second limiting convex ring 33 is located inside a gap formed by two ends of the first limiting convex ring 32. By restricting a rotation range of the second limiting convex ring 33 in the gap, a rotation angle of the main imaging base 21 is constrained. It should be noted that the limiting structure of this embodiment is only one implementation. Another type of limiting structure/device can also be used to restrict the rotation angle of the main imaging base 21, to prevent unconstrained 360° free rotation of the main imaging base 21, thus avoiding damage to or mis-operation on the imaging head caused by excessive rotation in an operation process, ensuring that each rotation is within a safe and controllable range, and further enhancing the stability and service life of the device.

In this embodiment, as shown in FIG. 1, FIG. 3, FIG. 4, and FIG. 7, the connection arms 212 downwards slantways extend respectively from the two sides of the main imaging base 21. As shown in FIG. 3 and FIG. 4, the main imaging base 21 and the elastic clamping portions 24 enclose an imaging region 213. At least two groups of cameras 23 are provided and are disposed around the imaging region 213 on the main imaging base 21 and the connection arms 212. The cameras 23 are connected to the control circuit board 12 through data wires provided, to implement image transmission.

The connection arms 212 of this embodiment are inclined with respect to the main imaging base 21. The camera 23 can be mounted on the main imaging base 21 and the connection arms 212, especially on the same horizontal plane projection. Two adjacent cameras 23 are disposed at an angle θ. A quantity of cameras 23 can be set as needed, and at least two groups of cameras are provided. In this embodiment, three groups of cameras 23 are taken as an example for description. As shown in FIG. 7, in this implementation, the three groups of cameras 23 are mounted on an inner wall of the main imaging base 21 and the two connection arms 212 respectively. The camera 23 of the main imaging base 21 is vertically oriented towards the imaging region 213, and the cameras 23 on the two connection arms 212 are also perpendicular to the connection arms 212, so that the cameras diagonally face two sides of the imaging region 213 respectively. In this way, the cameras 23 mounted on the connection arms 212 on the two sides form angles with an arrangement direction of the camera 23 on the main imaging base 21. For example, in FIG. 7, the camera 23 of the main imaging base 21 and the camera 23 mounted on the connection arm 212 on the right side are taken as an example. A central axis of the camera 23 disposed on the main imaging base 21 is A1, and a central axis of the camera 23 disposed on the connection arm 212 on the right side is A2. An angle of intersection between the central axis A1 and the central axis A2 is θ. A size of the angle θ can be set to 1°, 5°, 10°, 15°, 20°, 25°, 30°, 35°, 40°, 45°, 50°, 55°, 60°, 65°, 70°, 75°, 80°, 85°, or 90°. The angle can be selected based an actual need and controlled within a range of θ ≤ 90°. Therefore, imaging regions of the cameras 23 on the two connection arms 212 can overlap an imaging region of the camera 23 on the main imaging base 21 on left and right. This overlapping manner for the imaging regions can ensure that the imaging region 213 is completely covered, and teeth placed within the imaging region 213 are covered in a plurality of directions to the comprehensiveness and accuracy of imaging. Through this design, the imaging quality is enhanced, the operation process is also simplified, a diagnostic error caused by a viewing-angle blind spot is reduced, and a more reliable image support is provided for dental medical treatment. The movable arrangement of the connection arms 212 can assist in strengthening the function of the elastic clamping portions 24. Since the thickness of the alveolar bone dynamically changes, and a thickness of the alveolar bone varies at different positions. At a posterior tooth, the alveolar bone is wider, which will stretch and open the movable connection arms 212 and the elastic clamping portions 24, so that the angles between the connection arms 212 and the main imaging base 21 become larger. At an anterior tooth, the alveolar bone is relatively narrow, the connection arms 212 and the elastic clamping portions 24 are closer to an alveolar cavity, and the angles between the movable connection arms 212 and the main imaging base 21 become smaller. The cameras 23 are fixed on the connection arms 212. As the angles between the connection arms 212 and the main imaging base 21 change, the angle between the cameras also changes to an extent, thus dynamically adjusting the angle θ between the center axis A1 and the central axis A2 of the cameras to adapt to differences in different tooth positions. While ensuring that the imaging region 213 is always completely covered, it optimizes an overlapping range and matching precision of multi-angle imaging. This further enhances the accuracy and stability of three-dimensional reconstruction.

In the inclining arrangement between the connection arms 212 and the main imaging base 21, it is preferred that the connection arms 212 are fixedly connected to the main imaging base 21, and the connection arms 212 are stably connected to the main imaging base 21 through an integrated molding process or other fixing structures, thus ensuring the structural rigidity and the angle stability during long-term use. Meanwhile, this fixing manner effectively avoids an imaging deviation caused by mechanical looseness, thus further ensuring accurate matching and fusion of multi-angle images.

In other implementations, the connection arms 212 and the main imaging base 21 can also be connected in a movable connection manner. For example, angle adjustment is implemented through a hinge structure or a rotating shaft.

In this embodiment, since the connection arms 212 are inclined with respect to the main imaging base 21, in FIG. 7, when the camera 23 in the middle is vertically fixedly connected to the main imaging base 21, the cameras 23 on the connection arms 212 can also be directly vertically fixedly connected to the connection arms 212. Certainly, mounting angles of the camera 23 can also be adjusted, as long as the angle θ between two adjacent cameras 23 on the same horizontal plane projection is less than or equal to 90°.

Certainly, in some implementations, by maintaining the vertical mounting of the camera 23 in the middle and changing the tilting angles of the connection arms 212, the cameras 23 can achieve coverage in different viewing angles in an oral structure, and the optimized imaging quality can also be achieved. In addition, the connection arms 212 are made of a high-strength material. This ensures the structural stability, enhances the durability of the device, and ensures that accurate imaging performance can still be maintained during long-time use.

For another example, mounting positions of the cameras 23 can be flexibly adjusted. Capturing ranges and angles of the cameras 23 can be adjusted at different positions on the connection arms 212, so as to adapt to differences in oral structures of different patients and ensure that keycap details can be accurately captured in each imaging. This flexibility not only enhances the applicability of the device, but also further improves the diagnosis and treatment efficiency and accuracy and provides a powerful auxiliary tool for dentists.

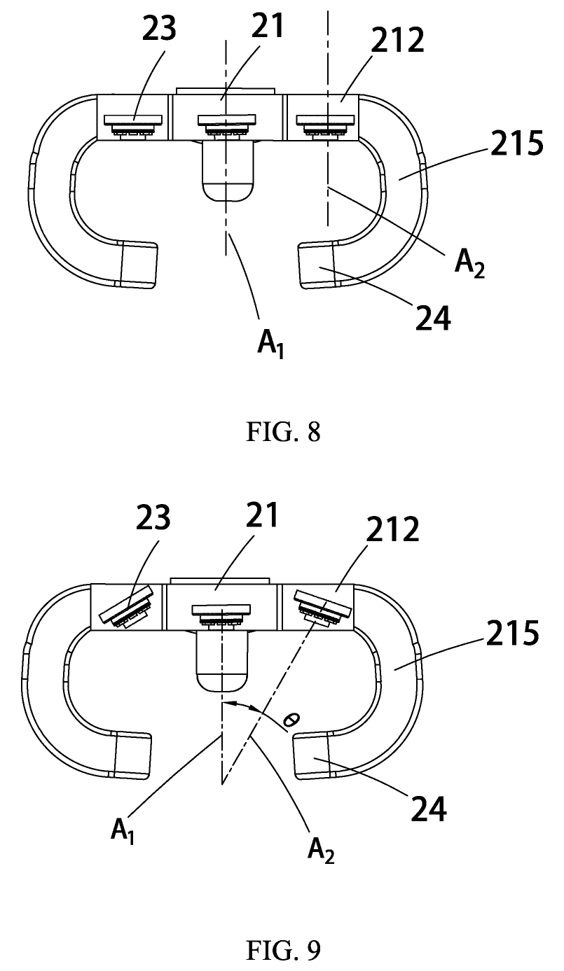

In addition, certainly, in other implementations, as shown in FIG. 8, the connection arms 212 can be arranged on two sides of the main imaging base 21 in parallel. Three groups of cameras 23 are also taken as an example. One group of cameras 23 is disposed on the main imaging base 21, and the other two groups of cameras 23 are respectively disposed on the connection arms 212. The three groups of cameras 23 are vertically downward. In this way, the three groups of cameras 23 are disposed in parallel, so that a spacing between two adjacent cameras needs to be controlled to satisfy overlapping of photographing regions between every two cameras and ensure a coverage range. Certainly, as shown in FIG. 9, the two groups of cameras 23 on the connection arms 212 can also tilt. The cameras 23 can tilt through fitness between a tilting groove body and a buckle. Thus, it can cause the capturing ranges of the three groups of cameras 23 to overlap and ensure that teeth are covered in all directions, without blind spots, thus ensuring the comprehensiveness and accuracy of imaging.

LED light groups 4 are arranged at a periphery the main imaging base 21 and/or peripheries of the cameras 23. The arrangement of the LED light groups 4 is mainly to improve a field-of-view environment of an oral cavity during operation. Specifically, as shown in FIG. 6, for the mounting of the cameras 23 in this embodiment, a mounting groove body 214 is formed in the main imaging base 21 or each connection arm 212. The cameras 23 are fixedly connected to the camera housing 231. The LED light groups 4 are arranged around the peripheries of the cameras 23 to ensure a lighting environment inside the oral cavity. In other implementations, the LED light groups 4 can also be separated from the cameras 23. For example, the LED light groups 4 can be uniformly placed on the main imaging base 21 to provide sufficient lighting for the cameras 23 and an inside of the oral cavity during operation.

The brightness of the LED light groups 4 can be adjusted to meet different oral environments and an observation need. The cameras 23 and the LED light groups 4 are both connected to the control circuit board 12 by using an FPC flexible flat cable 232, to ensure stable and efficient signal transmission.

As shown in FIG. 3 and FIG. 4, first bent portions 215 are arranged between the elastic clamping portions 24 and the connection arms 212, to cause the two elastic clamping portions 24 to be horizontally opposite to each other and form, between the two elastic clamping portions 24, a clamping opening 241 for passing through an alveolar cavity. The first bent portions 215 and the elastic clamping portions 24 are made of silicone or other elastic materials, so that the size of the clamping opening 241 can be self-adjusted based on the width of the alveolar bone when the alveolar bone is clamped. The adaptive fitting characteristic of the elastic clamping portions 24 further enhances the applicability and stability of the device in different oral shapes, so that the optimal imaging quality can be achieved in each examination.

As shown in FIG. 3 and FIG. 4, two groups of supports 5 are also arranged on the main imaging base 21, and the two groups of supports 5 are vertically arranged on the main imaging base 21, one in front and one behind. During use, the supports can resist against top surfaces of teeth, as a height limitation, to ensure the stability of a relative position. Physical marks and standard reference substances are provided on the supports 5, and physical marks and standard reference substances can also be provided on the elastic clamping portions 24. Certainly, the physical marks and the standard reference substances are arranged on either the supports or the elastic lamping portions.

Preferably, an elastic abutment portion can be further arranged at a bottom of the support 5. The elastic abutment portion can be made of a silicone material or other elastic materials with the same effect, so that the support can slide on tooth surfaces more smoothly.

It should be noted that arrangement positions of the physical marks and the standard reference substances can be flexibly set. Generally, the physical marks and the standard reference substances can be disposed at positions that can be observed from the outside during operation of an oral endoscope, which facilitates operations by doctors. A physical mark with a known length (such as a portion of a standard scale) is integrated onto the above assembly and is ensured to be clearly visible, so that precise size and color calibration can be provided for model reconstruction, thus enhancing the diagnostic reliability. It is convenient for quick positioning by a doctor using the physical mark, and consistent reference can also be provided during multi-angle imaging.

The above describes the preferred embodiments of the present disclosure and is not intended to limit the present disclosure. Any modification, equivalent replacement, and improvement made within the spirit and scope of the present disclosure shall fall within the protection scope of the present disclosure.

What is claimed is:

1. An oral endoscopic scanning device, comprising:
a handheld assembly, wherein a control circuit board is disposed within the handheld assembly;
an imaging head, wherein an imaging head comprises a main imaging base, at least two groups of cameras, and two elastic clamping portions respectively arranged on two sides of the main imaging base; connection arms are disposed between the main imaging base and the elastic clamping portions; the cameras surround an imaging region and are disposed on the main imaging base and/or the connection arms; on the same horizontal plane projection, two adjacent cameras are disposed at an angle, so that capturing ranges of the two adjacent cameras partially overlap, and after overlapping, the capturing ranges of all the cameras cover the imaging region; the cameras are connected to the control circuit board through data wires provided, to implement image transmission; and
a turning structure, wherein the main imaging base is connected to the handheld assembly through the turning structure to implement multi-angle rotation of the imaging head.

2. The oral endoscopic scanning device according to claim 1, wherein an angle θ between the two adjacent cameras on the same horizontal plane projection is less than or equal to 90°.

3. The oral endoscopic scanning device according to claim 2, wherein the connection arms are arranged on the two sides of the main imaging base in parallel, or the connection arms downwards slantways extend respectively from the two sides of the main imaging base.

4. The oral endoscopic scanning device according to claim 3, wherein first bent portions are arranged between the elastic clamping portions and the connection arms, to cause the two elastic clamping portions to be horizontally opposite to each other and form, between the two elastic clamping portions, a clamping opening for passing through an alveolar cavity.

5. The oral endoscopic scanning device according to claim 1, wherein light-emitting diode (LED) light groups are arranged at a periphery of the main imaging base and/or peripheries of the cameras.

6. The oral endoscopic scanning device according to claim 1, wherein the imaging head further comprises a connector; a connection slot with an opening in one side is formed in the main imaging base; the connector is plugged into the connection slot for locking; and the turning structure is arranged on the connector.

7. The oral endoscopic scanning device according to claim 6, wherein the turning structure comprises a connection ring body protruding from the connector; a connection through hole is formed in one end of the handheld assembly away from the control circuit board; the connection ring body passes through and is connected into the connection through hole in a threaded manner, to implement rotation of the imaging head.

8. The oral endoscopic scanning device according to claim 7, wherein the turning structure further comprises a limiting structure; the limiting structure comprises a first limiting convex ring disposed at a periphery of the connection through hole; and a second limiting convex ring fitting with the first limiting convex ring is also arranged on one side of the connection ring body.

9. The oral endoscopic scanning device according to claim 1, further comprising a support, wherein the support is perpendicularly arranged on the main imaging base.

10. The oral endoscopic scanning device according to claim 9, wherein a physical mark and a standard reference substance are arranged on the support and/or the elastic clamping portion.

11. The oral endoscopic scanning device according to claim 6, wherein the connection slot is of a dovetail joint structure; the connector is also configured as a dovetail joint structure; and the connector is plugged into the connection slot to implement stable connection.

12. The oral endoscopic scanning device according to claim 3, wherein the main imaging base is fixedly connected to the connection arms.

13. The oral endoscopic scanning device according to claim 3, wherein the main imaging base is movably connected to the connection arms.

14. The oral endoscopic scanning device according to claim 8, wherein the first limiting convex ring and the second limiting convex ring are located on a coaxial arc, and the second limiting convex ring is located inside a gap formed by two ends of the first limiting convex ring.

15. The oral endoscopic scanning device according to claim 5, wherein the cameras are fixedly connected to a camera housing; and the LED light groups are arranged around the peripheries of the cameras.

16. The oral endoscopic scanning device according to claim 5, wherein the LED light groups are separated from the cameras; and the LED light groups are uniformly arranged on the main imaging base to supply sufficient lighting for inside of an oral cavity during operation of the cameras.

17. The oral endoscopic scanning device according to claim 1, wherein the control circuit board is integrated with a Type-C interface and a wireless module.

18. The oral endoscopic scanning device according to claim 4, wherein the first bent portions and the elastic clamping portions are made of an elastic material.

19. The oral endoscopic scanning device according to claim 10, wherein an elastic abutment portion is further arranged at a bottom of the support.

20. The oral endoscopic scanning device according to claim 19, wherein the elastic abutment portion is made of an elastic material.
